(19) United States
(12) Patent Application Publication
(10) Pub. No.: US 2022/0254475 A1
(43) Pub. Date: Aug. 11, 2022

(54) METHOD AND APPARATUS FOR CONTROLLING NUTRITIONAL CONSUMPTION

(71) Applicant: Minji Koo, Frisco, TX (US)

(72) Inventor: Minji Koo, Frisco, TX (US)

(21) Appl. No.: 17/092,183

(22) Filed: Nov. 6, 2020

(51) Int. Cl. G16H 20/60; G09B 19/00

(52) U.S. Cl. CPC G16H 20/60; G09B 19/0092

(57) ABSTRACT

A method and apparatus of an electronic device is provided. The method and apparatus comprises of obtaining information of a first item; determining the information of the first item; saving the information of the first item in a memory; determining whether the first item includes a nutritional restriction based on the information of the first item; identifying, based on the information of the first item, information of a second item based on a determination that the first item includes the nutritional restriction, wherein the information of the second item is stored in the memory; and displaying the information of the second item including information indicating a conflict of nutritional consumption with the first item.

### CROSS-REFERENCE TO RELATED APPLICATIONS AND CLAIM OF PRIORITY

The present application claims priority to U.S. Provisional Patent Application Ser. No. 62/931,804 filed on Nov. 7, 2019. The content of the above-identified patent documents is incorporated herein by reference.

### TECHNICAL FIELD

This disclosure generally relates to an electronic device and method for controlling nutritional consumption. More specifically, this disclosure relates to an electronic device and method for controlling nutritional consumption based on the human's body condition, disease, confliction of nutrition, and more.

### BACKGROUND

Having control over the nutritional meals a person eats is a crucial part of survival. Having a good diet and being able to regulate what goes into the body promotes a healthy lifestyle including, but not limited to, prevention of a chronic or acute disease, cure of a condition, maintenance over one's weight, and protection for a healthy baby. Food, being one of the most important needs for any life, brings energy to the body to be physically active, emotionally stable, and psychologically conscious to surroundings. Without food, the human body could essentially collapse resulting in negative reactions that could leave serious, permanent conditions and even be fatal. As important as it is to eat, knowing how and what to eat is just as important.

### SUMMARY

This disclosure provides an electronic device and method for controlling nutritional consumption based on human's body condition, disease, confliction of nutrition, and more.

In one embodiment, an electronic device for controlling nutritional consumption is provided. The electronic device comprises a memory, a display, an input readable circuit, operably connected to the display and the memory, configured to obtain information of a first item. The electronic device further comprises a processor operably connected to the memory, the display, and the input readable circuit, the processor configured to: determine, through the input readable circuit, the information of the first item; save the information of the first item in the memory; determine whether the first item includes a nutritional restriction based on the information of the first item; identify, based on the information of the first item, information of a second item based on a determination that the first item includes the nutritional restriction, wherein the memory includes the information of the second item; and display, on the display, the information of the second item including conflict of nutritional consumption with the first item.

In another embodiment, a method of an electronic device for controlling nutritional consumption is provided. The method comprises: obtaining information of a first item; determining the information of the first item; saving the information of the first item in a memory; determining whether the first item includes a nutritional restriction based on the information of the first item; identifying, based on the information of the first item, information of a second item based on a determination that the first item includes the nutritional restriction, wherein the information of the second item is stored in the memory; and displaying the information of the second item including conflict of nutritional consumption with the first item.

In yet another embodiment, a non-transitory computer-readable medium comprising program code is provided. The non-transitory computer-readable medium comprising program code, that when executed by at least one processor, causes an electronic device to: obtain information of a first item; determine, through an input readable circuit, the information of the first item; save the information of the first item in a memory; determine whether the first item includes a nutritional restriction based on the information of the first item; identify, based on the information of the first item, information of a second item based on a determination that the first item includes the nutritional restriction, wherein the information of the second item is stored in the memory; and display, on a display, the information of the second item including conflict of nutritional consumption with the first item.

Other technical features may be readily apparent to one skilled in the art from the following figures, descriptions, and claims.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The term “ranging,” as well as derivatives thereof, mean that the fundamental measurements for ranging between devices are achieved by a transmission and a reception of one or more messages. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of this disclosure, reference is made to the following description, taken in conjunction with the accompanying drawings, in which:

FIG. 1 illustrates a flowchart of a method for controlling nutritional consumption operation according to embodiments of the present disclosure;

FIG. 2 illustrates an example electronic device for controlling nutritional consumption according to embodiments of the present disclosure;

FIG. 3 illustrates another example electronic device for controlling nutritional consumption according to embodiments of the present disclosure;

FIG. 4 illustrates yet another example electronic device for controlling nutritional consumption according to embodiments of the present disclosure;

FIG. 5 illustrates yet another example electronic device for controlling nutritional consumption according to embodiments of the present disclosure;

FIG. 6 illustrates an example network configuration for controlling nutritional consumption according to embodiments of the present disclosure;

FIG. 7 illustrates an example electronic device for controlling nutritional consumption according to embodiments of the present disclosure; and

FIG. 8 illustrates a flowchart of a method for controlling nutritional consumption according to embodiments of the present disclosure.

### DETAILED DESCRIPTION

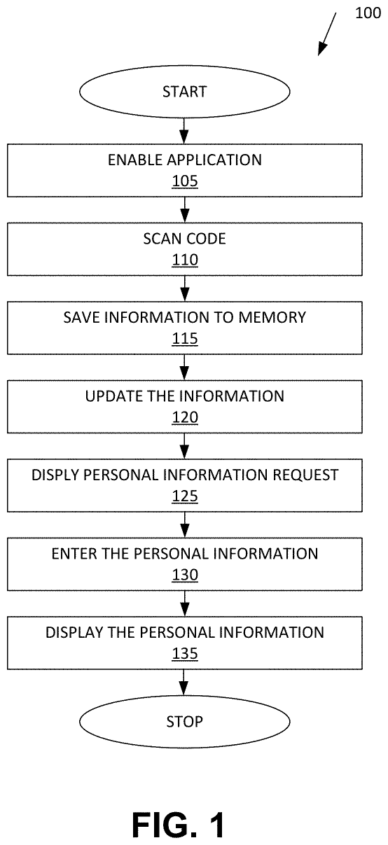

FIGS. 1 through 8, described below, and the various embodiments used to describe the principles of the present disclosure are by way of illustration only and should not be construed in any way to limit the scope of the invention. Those skilled in the arts will understand that the principles of the present invention may be implemented in any type of suitably arranged device or system.

Food, being one of the most important needs for any life, brings energy to the body to be physically active, emotionally stable, and psychologically conscious to surroundings. Without food, the human body could essentially collapse resulting in negative reactions that could leave serious, permanent conditions and even be fatal. As important as it is to eat, knowing how and what to eat is just as important. There are several ways to control one's diet: formulating a diet plan, learning the “good” and “bad” foods, limiting foods too high in sugar, preventing consuming the nutrition and/or food that has conflict each other, and/or even visiting a dietician.

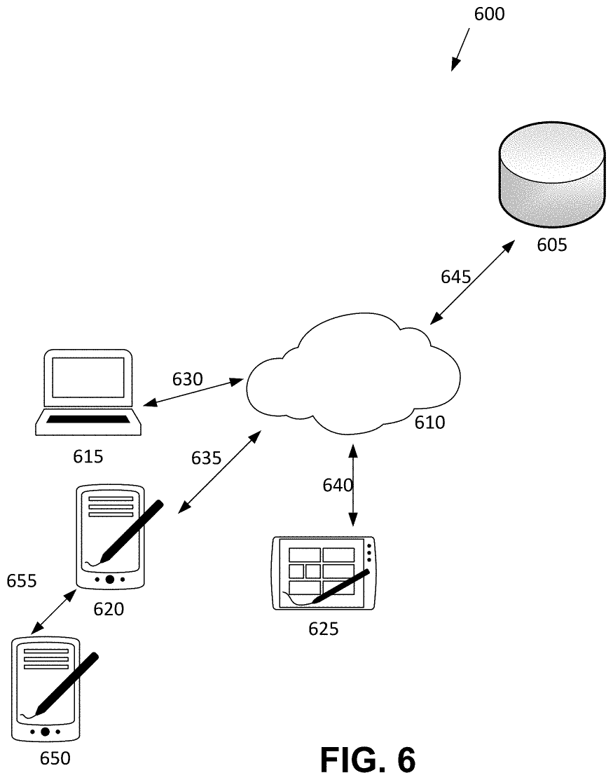

FIG. 1 illustrates a flowchart of a method 100 for controlling nutritional consumption operation according to embodiments of the present disclosure, as may be performed by an electronic device (e.g., 615, 620, 625 as illustrated in FIG. 6). The embodiment of the method 100 illustrated in FIG. 1 is for illustration only, and the method 100 could have the same or similar configuration. However, the electronic device may come in a wide variety of configurations, and FIG. 1 does not limit the scope of this disclosure to any particular implementation of an electronic device. An electronic device may include a portable electronic device or fixed electronic device.

In one embodiment, as illustrated in FIG. 1, the method 100 begins at step 105. In step 105, an electronic device enables an application on a display of the electronic device by opening/executing the application. In step 105, the electronic device enables an application that is installed in the electronic device. In one embodiment, the application may be executed by a user of the electronic device. In one example, a user may enable the application by touching an icon or a function, displayed on the display of the electronic device, that may execute the application in the electronic device.

In step 110, the electronic device may scan, as executed by the user, a quick response (QR) code, a bar code, or any input method including information of nutrition on a product by capturing a picture or any character of the code or character. Herein after, the QR code or the bar code inherently includes any input method for simplifying the description of the operation. And the reading, scanning, and recognizing (the QR code or the bar code) are commonly used each other. And the reading, scanning, and recognizing include any method to capture or obtain information of an object to be scanned, recognized, or read by the input method and/or device (e.g., a scanner, a QR code reader, a bar code reader, a camera).

In step 110, the QR code, the bar code, or any input method can be scanned, recognized, or read by a camera or any interface that may read or recognize the QR code, the bar code, or any input method. At least one readable interface (e.g., a QR code reader, a bar code reader, or any input method reader) is equipped in the electronic device. In one embodiment, any electronic external devices that are connected to the electronic device may be used for scanning at least one of the QR code, the bar code, or input method. In such embodiment, at least one external electronic device and the electronic device may be connected via a wireless communication protocol or a wired communication protocol using a wireless connection or a wired connection. In one embodiment, any mark, symbol, or sign including information of nutrition may be used for scanning operation instead of the QR code or the bar code.

In step 115, the electronic device may save the scanned nutritional information into a memory (e.g., database) that is equipped into the electronic device (e.g., 615, 620, 625 as illustrated in FIG. 6), or the external electronic device (e.g., 650 as illustrated in FIG. 6) that is wireless or wiredly connected to the electronic device, or a data base (e.g., 605 as illustrated in FIG. 6) through a network (e.g., 610 as illustrated in FIG. 6). In one example, a user may save the scanned nutritional information into at least one of the memory (database) of the electronic device (615, 620, 625), the external electronic device (650), or the databased (605) through the network (610), as illustrated in FIG. 6. In one example, the database may be implemented in the memory of the electronic device or in a network including an external database (e.g., 605 as illustrated in FIG. 6). The scanned nutritional information may be manually or automatically saved by the electronic device or the user.

In one embodiment, the database is implemented in the electronic device as a memory or in a network entity that is remotely located in a network or connected to the network as an independent network entity. In step 120, the electronic device may update the information that is saved on the memory (e.g., database). In one embodiment, the information may be updated based on existing information that has been saved on the database (e.g., in a form of memory) that is equipped in at least one of the electronic device, the external electronic device, or the data base through the network. In one embodiment, the information may be updated with replacing existing information save on the at least one of the electronic device, the external electronic device, or the data base through the network.

In step 125, the electronic device may display, on a screen of the electronic device or a screen of the external electronic device, a window requesting personal information of a user such as an age, a sex, or a medical history including diabetes, allergy symptoms, or user's any personal information etc. In one embodiment, the information requested by the electronic device may be displayed on the same window that is used for the previous displaying or another window that is different than the window displaying the previous information. In one embodiment, the information may be displayed on the external electronic device.

In step 130, the electronic device processes the personal information including user's body condition (e.g., for pregnancy, for diabetes, for any disease of the user, allergies, for general purposes etc.). In one embodiment, a user of the electronic device may enter her/his personal information (e.g., age, sex, and/or medical history including diabetes or allergy symptoms). In one embodiment, the personal information may have multiple conditions and/or combination of at least two conditions that may give medical symptoms or concerns to the user of the electronic device. In one embodiment, the personal information may be entered into the same window or a new window that is different than the widow displaying the previous information.

In step 135, the electronic device may display the personal information in a responding window to confirm all fields are accurate and correct. In one embodiment, the personal information may be displayed on the same window or a new window that may be different than the window displaying the previous information.

TABLE 1
Alarming foods for diabetics

| | NUTRITIONAL | |
|---|---|---|
| NAME OF FOOD | ELEMENTS | REASONING |
| SODA | Carbohydrates, | Insulin Resistant |
| | Fructose | |
| DRIED FRUITS | Carbohydrates, Sugar | Raise Blood Sugar Levels |
| FRENCH FRIES | Carbohydrates, Fiber | Raise Blood Sugar Levels |
| GRAINS | Carbohydrates | Insulin Resistant |

As illustrated in TABLE 1, a personal information input can be used to formulate a correct diet plan for a person or a person who uses the electronic device as a user of the application. TABLE 1 shows an example of a diabetic patient's brief alarming food list. As shown in FIG. 1, by inputting the personal information, the application may decipher the information and be aware of any alarming foods for the user. TABLE 1 is an example for a diabetic patient. TABLE 1 does not limit any scope of the present disclosure or any implementation of the present disclosure.

TABLE 2
Alarming foods for pregnant women

| | NUTRITIONAL | |
|---|---|---|
| NAME OF FOOD | ELEMENTS | REASONING |
| FISH | Mercury | Developmental Problems |
| COFFEE | Caffeine | Limit Fetal Growth |
| JUNK FOOD | Fats, Calories, Sugars | Overweight Children |
| ALCOHOL | Alcohol | Fetal Alcohol Syndrome |

As illustrated in TABLE 2, a personal information input can be used to formulate a correct diet plan for one. TABLE 2 shows an example of a pregnant women's brief alarming food list. As shown in FIG. 1, by inputting personal information, the application may decipher the information and be aware of any alarming foods for the user. TABLE 2 is an example for a pregnant woman. TABLE 2 does not limit any scope of the present disclosure or any implementation of the present disclosure.

TABLE 3
Alarming foods for vegetarians/vegans

| | NUTRITIONAL | |
|---|---|---|
| NAME OF FOOD | ELEMENTS | REASONING |
| GELATIN | Meat | Processed from animal |
| | | tissue and bone |
| SHELLAC | Meat | Processed from insects |
| ALCOHOL | Meat | Processed with gelatin |
| MEAT | Meat | Processed from animal |
| | | tissue and bone |

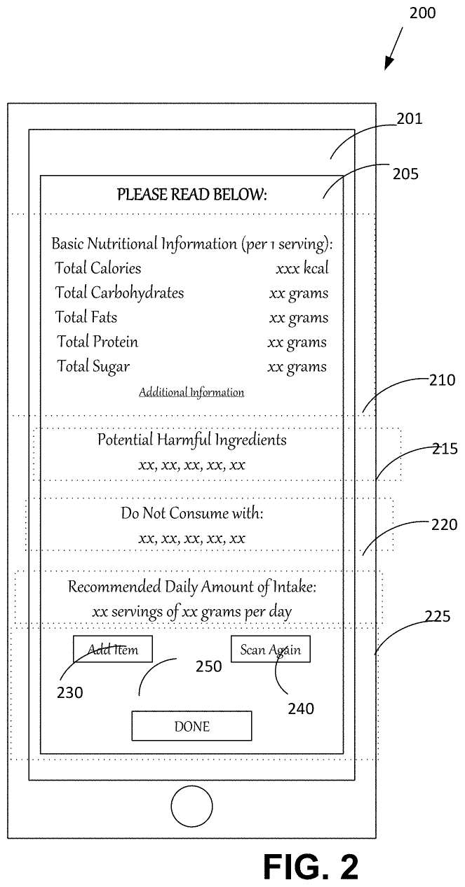

As illustrated in TABLE 3, nutritional information may pull up various names of food that may be an alarming for a user. As shown above in TABLE 3, vegan and vegetarian users may use this application as a reference to steer clear of non-vegan/non-vegetarian, plant-based diets. As shown in FIG. 2, nutritional facts may be displayed in which the user can judge whether to consume or not consume the product. TABLE 3 is an example of a vegetarian and/or vegan diet. TABLE 3 does not limit any scope of the present disclosure or any implementation of the present disclosure.

TABLE 4
Alarming foods for young children

| | NUTRITIONAL | |
|---|---|---|
| NAME OF FOOD | ELEMENTS | REASONING |
| HONEY | Bacterium | Toxic Spores |
| COFFEE | Caffeine | Development Issues |
| JUNK FOOD | Fats, Calories, Sugars | Overweight Children |
| CHOCOLATE | Sugars, Caffeine | Overweight Children, |
| | | Diabetes Risk, |
| | | Developmental Issues |

As illustrated in TABLE 4, nutritional information may pull up various names of food that may be an alarming for a user. As shown above in TABLE 4, children users including young children may use this application as a reference to steer clear of unhealthy diets. As shown in FIG. 2, nutritional facts may be displayed in which the user can judge whether to consume or not consume the product. TABLE 4 is an example of a young child's diet. TABLE 4 does not limit any scope of the present disclosure or any implementation of the present disclosure.

TABLE 5
Alarming foods for elderly

| | NUTRITIONAL | |
|---|---|---|
| NAME OF FOOD | ELEMENTS | REASONING |
| SOFT CHEESE | Acidic | Food-Poisoning |
| | | (Listeria) |
| RAW EGG | Chemicals | Weakens Immune |
| | | System |
| BEAN SPROUTS | Chemicals | Food-Poisoning |
| CITRIC FRUITS | Citric Acid | High Blood Pressure, |
| | | Drug Effects |

As illustrated in TABLE 5, nutritional information may pull up various names of food that may be an alarming for the user. As shown above in TABLE 5, elderly users may use this application as a reference to steer clear of unhealthy diets. TABLE 5 does not limit any scope of the present disclosure or any implementation of the present disclosure.

FIG. 2 illustrates an example electronic device 200 (e.g., 615, 620, 625 as illustrated in FIG. 6) and external electronic device (e.g., 650 as illustrated in FIG. 6) for controlling nutritional consumption according to embodiments of the present disclosure. The embodiment of the electronic device 200 illustrated in FIG. 2 is for illustration only, and the electronic device 200 could have the same or similar configuration. However, the electronic device 200 may come in a wide variety of configurations, and FIG. 2 does not limit the scope of this disclosure to any particular implementation of an electronic device.

In one embodiment, as illustrated in FIG. 2, information, based on a human body condition, in a responding window 201 includes several information. A first section 205 of the responding window 201 shows the basic nutritional elements of the product that was scanned or recognized including at least one of total calories, total carbohydrates, total fats, total protein, or total sugars. As illustrated in FIG. 2, there is an “Additional Information” option which opens a new window of the same format with extra information including at least one of vitamins, iron percentage, minerals, or protein. The “Additional Information” may provide further information for the nutritional elements of the product. The second section 210 of the responding window 201 displays harmful ingredients based on a person's body condition (e.g., pregnancy, diabetes, dieses etc.). In one example, a user of the electronic device may input this information. A third section 215 of the responding window 201 displays the nutrition and/or food that may cause conflict with food scanned (e.g., recognized or read) by an input method circuit (e.g., camera 710, a scanner, a QR code reader etc.). A fourth section 220 of the responding window shows a recommended daily consumption amount in grams (or any unit of the amount such as ounce, pound, kilogram) per day (or a week, a month, or any length of a time period). A fifth section 225 of the responding window 201 gives three options for the user to choose from.

In one example, the first section 205, the second section 210, the third section 215, the fourth section 220, and the fifth section 225 may be displayed on the same window. In one example, at least two of the window sections (e.g., 205, 210, 220, 225) may be displayed in the same window. In one example, each of the window sections (e.g., 205, 210, 215, 220, 225) may be displayed individually on the different windows.

As illustrated in FIG. 2, the “Add Item” 230 option allows the user to scan or read a second item to save information of the second item into a memory of the electronic device. As illustrated in FIG. 2, the “Scan Again” 240 option allows the user to scan or read the item again to save information of the second item to the memory of the electronic device. As illustrated in FIG. 2, the “Done” 250 allows the user to finish and confirm scanning or reading all the information (of the second item) presented. Therefore, the user may choose good food and/or nutrition upon requested by the user's body condition.

As illustrated in FIG. 2, a number of windows is for illustration only, and the electronic device 200 could have the same or similar configuration including the number of windows. However, the electronic device 200 may come in a wide variety of configurations of the number of windows. FIG. 2 does not limit the scope of this disclosure to any particular implementation of an electronic device.

As illustrated in FIG. 2, some icons such as the “Add Item” 230, the “Scan Again” 240, and the “DONE” 250 are for illustration only, and the electronic device 200 could have the same or similar configuration including the icons. However, the electronic device 200 may come in a wide variety of configurations of the icons. FIG. 2 does not limit the scope of this disclosure to any particular implementation of an electronic device.

In one embodiment, the icons (e.g., 230, 240, 250) may be displayed on the screen of the electronic device (or an external electronic device) and a user may touch or click the icons to execute a function of the icon. In one embodiment, the icons (e.g., 230, 240, 250) may be implemented as physical buttons installed on the electronic device. In one embodiment, the icons (e.g., 230, 240, 250) may be implemented as an icon that may be touchable or clickable to execute a function of the icon on a screen of an external electronic device. In one embodiment, the icons (e.g., 230, 240, 250) may be implemented, as physical buttons installed on an external electronic device. In such embodiment, the external electronic device and the electronic device may be connected wirelessly or wiredly.

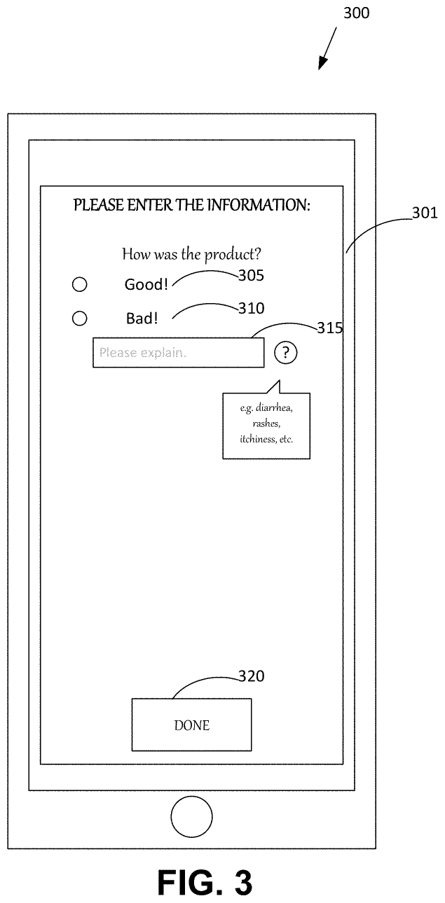

FIG. 3 illustrates another example electronic device 300 for controlling nutritional consumption according to embodiments of the present disclosure, as may be performed by an electronic device (e.g., 615, 620, 625 as illustrated in FIG. 6) or an external electronic device (e.g., 650 as illustrated in FIG. 6). The embodiment of the electronic device 300 illustrated in FIG. 3 is for illustration only, and the electronic device 300 could have the same or similar configuration. However, the electronic device may come in a wide variety of configurations, and FIG. 3 does not limit the scope of this disclosure to any particular implementation of an electronic device.

As illustrated in FIG. 3, an electronic device displays, on a display, some icons 305, 310, 315, 320. The electronic device may show a responding window 301. The icons 305 and 310 show “Good” and “Bad.” The electronic device displays “How was the product” and then displays the icon 305 and 310. Based on a user decision, the use may indicate “Good” (e.g., icon 305) or “Bad” (e.g., icon 310) by touching or clicking the icons. In one embodiment, the icons (e.g., 305, 310, 315, 320) may be displayed on the screen of the electronic device and a user may touch or click the icons to execute a function of the icon. In one embodiment, the icons (e.g., 305, 310, 315, 320) may be implemented as physical buttons installed on the electronic device. In one embodiment, the icons (e.g., 305, 310, 315, 320) may be implemented as an icon that may be touchable or clickable to execute a function of the icon, on an external electronic device. In one embodiment, the icons (e.g., 305, 310, 315, 320) may be implemented, as physical buttons installed on an external electronic device. In such embodiment, the external electronic device and the electronic device may be connected wirelessly or wiredly.

In one embodiment, the icon 315 may be used as an input window for a user. When the user has a concern or any memo that the user needs to recognize with the product, the user may enter symbols, characters, or language to describe the product in the icon 315. The icon 320 executes that the operation by touching or clicking the icon 305 or 310 has been finished.

In one example, the icons 305, 310, 315, and 320 may be displayed on the same window of the screen of the electronic device. In one example, at least two of the icons may be displayed in the same window of the screen of the electronic device. In one example, each of icons may be displayed individually on the different windows.

In one embodiment, as illustrated in FIG. 3, the user enters nutrition and/or food the user consumed, and the user may enter, in the icon 315 (e.g., input window), any symptoms if the user experienced any discomfort with the food the user consumed (e.g., diarrhea, stomach, headache, itching, alleging, or any abnormal body status). When the user scans a bar code, a character code, or a QR code of a product, by purchasing, an application running in the electronic device alarms, based on the information entered into the icon 315 (e.g., input window), that the user had experiences, possibly a number of causes, some uncomfortable state with the food and/or nutrition, including a specific symptom(s). Therefore, the user may avoid continuing to consume the product.

TABLE 6
Alarming foods for cancer patients

| | NUTRITIONAL | |
|---|---|---|
| NAME OF FOOD | ELEMENTS | REASONING |
| RED MEAT | Protein | Excessive byproduct |
| | | buildup |
| PICKLES | Nitrates | Carcinogenic |
| DEEP FRIED FOODS | Protein | Excessive Carcinogen |
| POULTRY | Protein | Excessive byproduct |
| | | buildup |

As illustrated in TABLE 6, alarming foods may be noted. As shown above in TABLE 6, an example of a cancer patient's alarming food list may show. The reasoning column on TABLE 6 may show based on the user's response (for example, user's body condition or the information entered into the window 315 as illustrated in FIG. 3). An example is shown in TABLE 6 for a cancer patient. TABLE 6 does not limit any scope of the present disclosure or any implementation of the present disclosure.

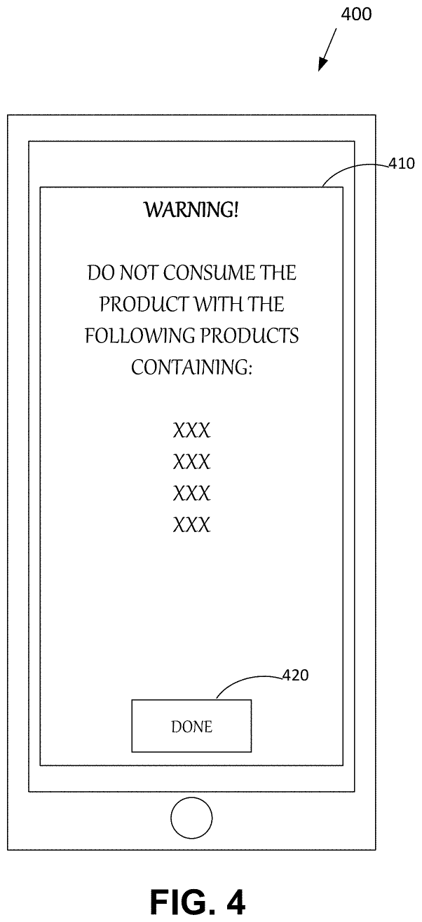

FIG. 4 illustrates yet another example electronic device 400 for controlling nutritional consumption according to embodiments of the present disclosure. The embodiment of the electronic device 400 illustrated in FIG. 4 is for illustration only, and the electronic device 400 could have the same or similar configuration. However, the electronic device may come in a wide variety of configurations, and FIG. 4 does not limit the scope of this disclosure to any particular implementation of an electronic device.

As illustrated in FIG. 4, an electronic device displays, on a display, a window 410. The window 410 may include information. The electronic device may display, for example, “DO NOT CONSUME THE PRODUCT WITH THE FOLLOWING PRODUCTS CONTAINING” and may display the name of items that were purchased or indicated by a user. In one embodiment, the electronic device may display the name of items (e.g., food, nutrition) that were saved on the memory (e.g., database).

In one embodiment, information included in the 410 may be displayed on the same screen of the electronic device or an external electronic device (e.g., 650 as illustrated in FIG. 6). In one embodiment, each information and the items may be displayed individually on the different screen of the electronic device.

In one embodiment, the information may be displayed on an external electronic device. In such embodiment, the external electronic device and the electronic device may be connected wirelessly or wiredly.

In one embodiment, the icon “DONE” 420 executes that the operation by showing the information has been finished.

In one example, the icon “DONE” 420 may be displayed on the same window 410 of the screen of the electronic device.

In one embodiment, as illustrated in FIG. 4, when the user scans a QR code or a bar code of a product the user is willing to buy, the application displays at least one food and/or nutrition that may conflict with other food and/or nutrition. For example, when the user buys spinaches, the application alarms the user that the spinaches may cause harm with milk and dairy products if the user consumes them together. In other words, the application displays a combination of foods and/or nutrition including a negative combination that may provide harmful action to a human body. Therefore, the user may avoid, based on the alarming information, purchasing or consuming items that contain certain products with the item they are consuming.

TABLE 7
Conflicting Food Combinations

| | NUTRITIONAL | |
|---|---|---|
| NAME OF FOOD | ELEMENTS | REASONING |
| SPINACH + DAIRY | Iron | Iron Deficiency |
| BEANS + RED WINE | Iron | Iron Deficiency |
| MELON + MOST | Acids | Digestive Problems |
| FOODS | | |
| Crab + Persimmon | Iron | Indigestion, food |
| | | poisoning |
| Fish (including higher | Fat | diarrhea |
| FAT) + Peach | | |
| Carrot + Cucumber + | Vitamin | Vitamin C deficiency |
| Radish | | |
| Wine + Curry | Alcohol | Pain on mouth |
| Chicken + Plum | Acids | Food poisoning |
| Shrimp + Tomato | Nitrite | Upset stomach |

As illustrated in TABLE 7, food combinations to be avoided may be displayed on the application. As shown in TABLE 7, as an example, certain food combinations are shown that are highly recommended to be avoided for consumption at the same time or same day. This may be displayed on the window as shown in FIG. 4. TABLE 7 does not limit any scope of the present disclosure or any implementation of the present disclosure.

As illustrated in FIG. 4, the electronic device may display a specific food or nutrition that may conflict with other food or foods, or nutrition. In one embodiment, when a user purchases a crab and scans a QR code or a bar code attached on the crab, the electronic device as illustrated in FIG. 4 may show “DO NOT CONSUME THE PRODUCTS WITH THE FOLLOWING PRODUCTS CONTAINING” and the electronic device may display, for example, a persimmon.

In one embodiment, a user scans the crab and the persimmon, the electronic device may display “DO NOT CONSUME THE CRAB WITH THE PERSIMMON.”

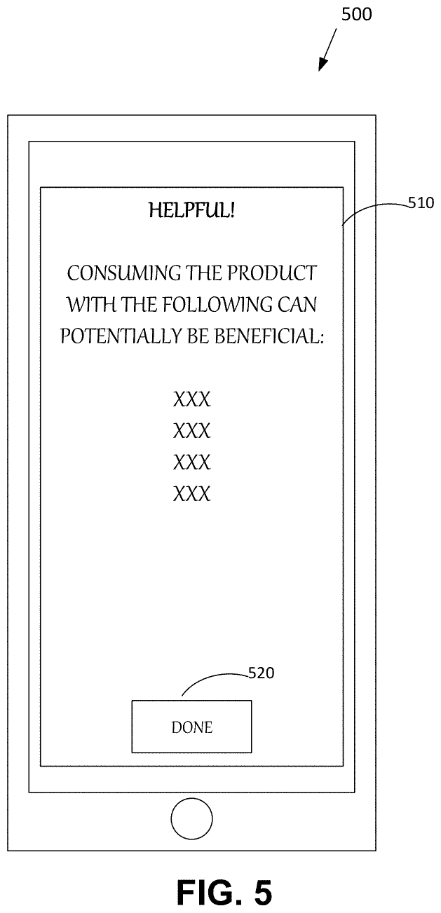

FIG. 5 illustrates yet another example an electronic device 500 for controlling nutritional consumption according to embodiments of the present disclosure. The embodiment of the electronic device 500 illustrated in FIG. 5 is for illustration only, and the electronic device 500 could have the same or similar configuration. However, the electronic device may come in a wide variety of configurations, and FIG. 5 does not limit the scope of this disclosure to any particular implementation of an electronic device.

As illustrated in FIG. 5, an electronic device displays, on a display, a window 510. The window 510 may include information. The electronic device may display, for example, “CONSUMING THE PRODUCT WITH THE FOLLOWING CAN POTENTIAL BE BENEFICIAL” and may display the name of items that were purchased or indicated by a user. In one embodiment, the electronic device may display the name of items (e.g., food, nutrition) that were saved on the memory (e.g., database).

In one embodiment, information included in the 510 may be displayed on the same screen of the electronic device. In one embodiment, each information and the items may be displayed individually on the different screen of the electronic device.

In one embodiment, the information may be displayed on an external electronic device. In such embodiment, the external electronic device and the electronic device may be connected wirelessly or wiredly.

In one embodiment, the icon “DONE” 520 executes that the operation by showing the information has been finished.

In one example, the icon “DONE” 520 may be displayed on the same window 510 of the screen of the electronic device.

In one embodiment, as illustrated in FIG. 5, when the user scans a QR code or a bar code of a product the user is willing to buy, the application displays at least one food and/or nutrition that may be positively acted with other food or foods, and/or nutrition. For example, when the user buys meat products, the application alarms that vegetables such as lettuce, romaine, and carrots may be positively balanced with the meat products if the user consumes them together. In other words, the application displays a combination of food or foods, and/or nutrition including a positive combination. Therefore, the user may actively pursue purchasing the two products such as the meat product and the vegetables.

FIG. 6 illustrates an example network configuration 600 for controlling nutritional consumption according to embodiments of the present disclosure. The embodiment of the network configuration 600 illustrated in FIG. 6 is for illustration only, and the network configuration 600 could have the same or similar configuration. However, the electronic device may come in a wide variety of configurations, and FIG. 6 does not limit the scope of this disclosure to any particular implementation of an electronic device.

In one embodiment, as illustrated in FIG. 6, the electronic devices (e.g., 615, 620, 625) may be remotely connected to a database 605, through over the air interface or wired interface using conventional wireless or wired communication system protocols (e.g., 630, 635, 640, 645), including information associated with the nutrition and/or food. The database 605 may be located in an entity of a communication network 610. In one example, the database 605 can be located out of the communication network 610 as shown in FIG. 6. In one example, the database 605 can be located in the communication network 610 as an entity of the communication network 610. This configuration is not shown in FIG. 6. An external electronic device 650 is connected with an electronic device 620. In one embodiment, the external electronic device 650 may be connected with the electronic device 615 or 625. In one example, the external electronic device 650 may be connected with two or more electronic devices (e.g., 615, 620, 625). A connection 655 between the external electronic device 650 and the electronic device 620 may comprise a wireless communication protocol (e.g., long term evolution (LTE), 4^{th} generation (4G) or 5G communication system, or beyond communication systems) or a wired communication protocol as illustrated in 630, 635, 640, 645.

As illustrated in FIG. 6, the electronic device (e.g., 615) may comprise personal laptops, tablet computers, and desktop computers. As illustrated in FIG. 6, the electronic device (e.g., 620) may comprise smartphones and personal digital assistants (PDAs). As illustrated in FIG. 6, the electronic device (e.g., 625) may comprise tablet personal computers (PCs) including iPads, Galaxy tablets, etc.

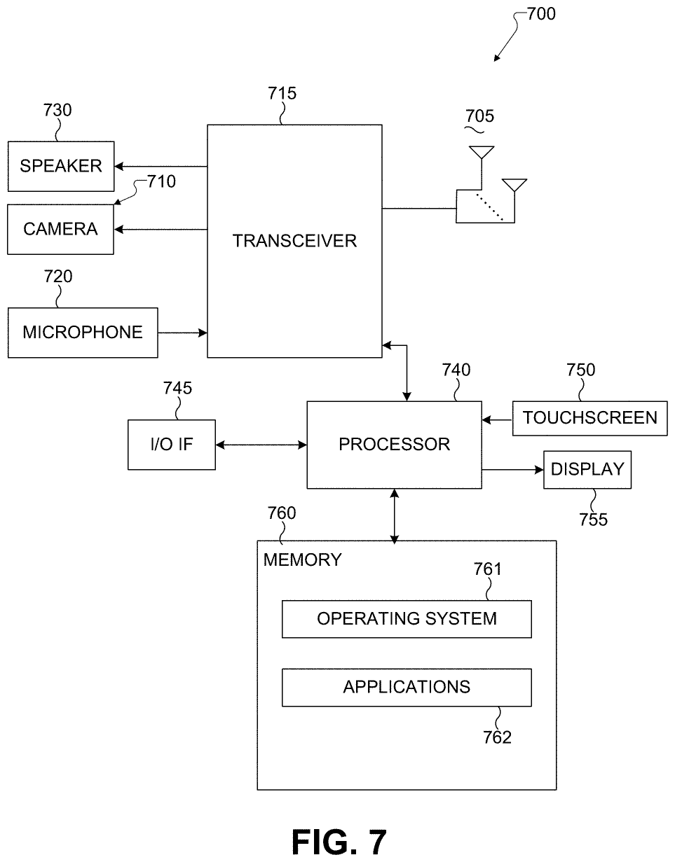

FIG. 7 illustrates an example electronic device 700 according to embodiments of the present disclosure. The embodiment of the electronic device 700 illustrated in FIG. 7 is for illustration only, and the electronic device 700 could have the same or similar configuration. However, the electronic device may come in a wide variety of configurations, and FIG. 7 does not limit the scope of this disclosure to any particular implementation of an electronic device.

As shown in FIG. 7, the electronic device 700 (e.g., 615, 620, 625, and 650 as illustrated in FIG. 6) includes a set of antennas 705, a transceiver 715, and a microphone 720. The electronic device 700 also includes a camera(s) 710, a speaker 730, a processor 740, and an input/output (I/O) interface (IF) 745, a touchscreen 750, a display 755, and a memory 760. The memory 760 includes an operating system (OS) 761 and one or more applications 762. In one embodiment, the one or more application comprises an application or a program as illustrated in the aforementioned embodiments and/or examples.

In one embodiment, the I/O IF 745 may include a QR code reader, a bar code reader, or any type of reader that may read, obtain, or recognize a symbol, a character, or a drawing.

In one embodiment, a barcode reader may reach out to a wide network of social barcoding and coding to find the exact or very similar products. Foods with no barcode such as homemade foods may require ingredients (only major) from a set list of ingredients given. In another embodiment, the set list of ingredients may be given but no large scope details may be included in the calculation of nutrition consumption.

In one embodiment, the camera 710 can read or scan the QR code or the bar code for controlling nutritional consumption as illustrated in the aforementioned embodiments and/or examples. In one embodiment, the camera 710 may include a program to read or scan the QR code or the bar code for controlling nutritional consumption as illustrated in the aforementioned embodiments and/or examples.

In one embodiment, the camera 710 may include a QR code reader, a bar code reader, or any type of reader that may read, obtain, or recognize a symbol, a character, or a drawing.

In one embodiment, the speaker 730 may translate the text that may be displayed on the touchscreen 750 or the display 755 into voice message for the user. In such embodiment, the user may hear the displaying information or read the information.

In one embodiment, the electronic device 700 transmits, to the database 605, the information (e.g., user related information or information entered by the user) using a wireless communication protocol or a wired communication protocol (e.g., 630, 635, 640, 645). In one embodiment, the electronic may receive, from the database via a wireless communication protocol and/or wired communication protocol (e.g., 630, 635, 640, 645), information corresponding to the information (e.g., user related information or information entered by the user).

The processor 740 can include one or more processors or other processing devices and execute the OS 761 stored in the memory 760 in order to control the overall operation of the electronic device 700. For example, the processor 740 could control the reception of signals by the transceiver 715. In some embodiments, the processor 740 includes at least one microprocessor or microcontroller.

The processor 740 is also capable of executing other processes and programs resident in the memory 760 (e.g., database), such as application for controlling nutritional consumption. The processor 740 can move data into or out of the memory 760 as required by an executing process. In some embodiments, the processor 740 is configured to execute the applications 762 based on the OS 761 or in response to signals to/from a database that may include data information for controlling nutritional consumption. The processor 740 is also coupled to the I/O interface 745, which provides the electronic device 700 with the ability to connect to other devices, such as laptop computers and handheld computers. The I/O interface 745 is the communication path between these accessories and the processor 740.

In one embodiment, the processor 740 may execute the application that is installed in the electronic device for controlling nutritional consumption. In one embodiment, the processor may process, by the electronic device or the user, any information, commands, instructions, and/or orders for controlling nutritional consumption.

The processor 740 is also coupled to the touchscreen 750 and the display 755. The operator of the electronic device 700 can use the touchscreen 750 to enter data into the electronic device 700. The display 755 may be a liquid crystal display, a light emitting diode display, or other display capable of rendering text and/or at least limited graphics, such as from web sites. In one embodiment, the display displays information as illustrated in FIGS. 2 through 5.

The memory 760 is coupled to the processor 740. Part of the memory 760 could include a random-access memory (RAM), and another part of the memory 760 could include a Flash memory or other read-only memory (ROM).

In one embodiment, the memory 760 may store the application for controlling nutritional consumption. In one embodiment, the memory may store the any information related to database, user information, or pre-determined information for controlling nutritional consumption. In one embodiment, the memory 760 may store the application as illustrated in FIGS. 2 through 5.

Although FIG. 7 illustrates one example of an electronic device 700, various changes may be made to FIG. 7. For example, various components in FIG. 7 could be combined, further subdivided, or omitted and additional components could be added according to particular needs. As a particular example, the processor 740 could be divided into multiple processors, such as one or more central processing units (CPUs) and one or more graphics processing units (GPUs). Also, while FIG. 7 illustrates the electronic device 700 configured as a mobile telephone or smartphone, the electronic device could be configured to operate as other types of mobile or stationary devices.

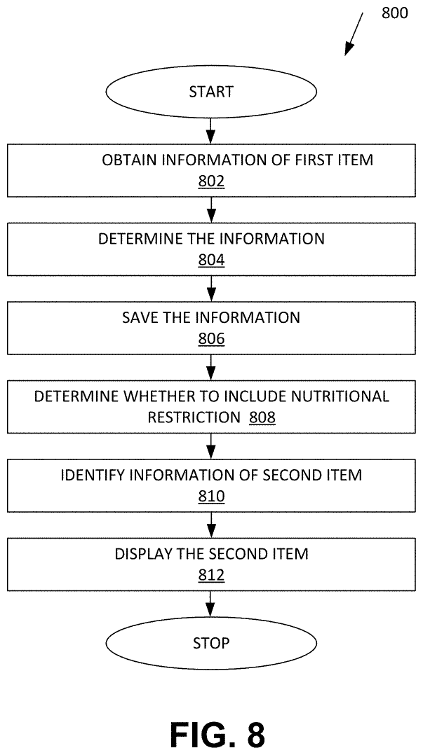

FIG. 8 illustrates a flowchart of a method 800 for controlling nutritional consumption according to embodiments of the present disclosure, as may be performed by an electronic device (e.g., 615, 620, 625, 650 as illustrated in FIG. 6). The embodiment of the method 800 illustrated in FIG. 8 is for illustration only, and the method 800 could have the same or similar configuration. However, the electronic device may come in a wide variety of configurations, and FIG. 8 does not limit the scope of this disclosure to any particular implementation of an electronic device.

As illustrated in FIG. 8, the method 800 begins at step 802. In step 802, an electronic device obtains information of a first item.

Subsequently, in step 804, the electronic device determines the information of the first item.

Subsequently, in step 806, the electronic device saves the information of the first item in a memory.

Subsequently, in step 808, the electronic device determines whether the first item includes a nutritional restriction based on the information of the first item.

Next, in step 810, the electronic device identifies, based on the information of the first item, information of a second item based on a determination that the first item includes the nutritional restriction, wherein the information of the second item is stored in the memory.

Finally, in step 812, the electronic device displays the information of the second item including conflict of nutritional consumption with the first item.

In one embodiment, the electronic device determines whether the first item includes, based on the information of the first item, a nutritional benefit based on the information of the first item, identifies, based on the information of the first item, the second item based on a determination that the first item includes the nutritional benefit, and displays, on the display, the information of the second item including information indicating the nutritional benefit for consuming with the first item.

In one embodiment, the electronic device identifies a body condition including at least one of an illness, a disease, a pregnancy, or a diabetes, determines whether the first item includes, based on the information of the first item, a nutritional restriction or benefit associated with the body condition based on the information of the first item, and displays, on the display, whether the first item includes information indicating the nutritional restriction or benefit of consumption for the body condition.

In one embodiment, the electronic device identifies a medicine being used for the body condition, determines whether the first item includes, based on the information of the first item, the nutritional restriction or benefit associated with the medicine based on the information of the first item; and displaying, on the display, whether the first item includes information indicating the nutritional restriction or benefit of consumption for the medicine.

In one embodiment, the electronic device identifies an age condition, determines whether the first item includes, based on the information of the first item, a nutritional restrictions or benefits associated with the age condition based on the information of the first item, and displays, on the display, whether the first item includes information indicating the nutritional restrictions or the benefits for the age condition.

In one embodiment, the electronic device identifies an eating habit, determines whether the first item includes, based on the information of the first item, a nutritional restriction for the eating habit based on the information of the first item, and displays, on the display, whether the first item includes information indicating the nutritional restriction for the eating habit.

In one embodiment, the electronic device determines whether the first item includes, based on the information of the first item, an intake cycle for consuming of the first item, and displays, on the display, information indicating the intake cycle of the first item for healthy consuming of the first item.

In one embodiment, the intake cycle may recommend one to take a break from a certain food item until all contents of the food are properly digested and excreted from the body. This intake cycle would be beneficial in determining whether one has an increased amount of a certain chemical or vitamin in their body which could rather harm than do benefits to their internal organs such as the pancreas.

In one embodiment, the electronic device stores at least one of a pre-determined body condition, a pre-determined age condition, or a pre-determined eating habit, stores at least one of a body condition, an age condition, or an eating habit being entered through an input circuit including at least one of a touchscreen or a key pad of the electronic device, obtains, through a camera, the information of the first and second items based on at least one of a QR code or a bar code; and obtains, through a QR code reader or a bar code reader, the information of the first and second items based on at least one of a QR code or a bar code associated with the information of the first and second items. The above flowcharts illustrate example methods that can be implemented in accordance with the principles of the present disclosure and various changes could be made to the methods illustrated in the flowcharts herein. For example, while shown as a series of steps, various steps in each figure could overlap, occur in parallel, occur in a different order, or occur multiple times. In another example, steps may be omitted or replaced by other steps.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claims scope. The scope of patented subject matter is defined by the claims.

The description in the present application should not be read as implying that any particular element, step, or function is an essential or critical element that must be included in the claim scope. The scope of patented subject matter is defined only by the allowed claims. Moreover, none of the claims is intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” or “controller” within a claim is understood and intended to refer to structures known to those skilled in the relevant art, as further modified or enhanced by the features of the claims themselves, and is not intended to invoke 35 U.S.C. § 112(f).

What is claimed is:

1. An electronic device for controlling nutritional consumption, the electronic device comprising:
a memory;
a display;
an input readable circuit, operably connected to the display and the memory, configured to obtain information of a first item;
a processor operably connected to the memory, the display, and the input readable circuit, the processor configured to:
determine, through the input readable circuit, the information of the first item;
save the information of the first item in the memory;
determine whether the first item includes a nutritional restriction based on the information of the first item;
identify, based on the information of the first item, information of a second item based on a determination that the first item includes the nutritional restriction, wherein the memory includes the information of the second item; and
display, on the display, the information of the second item including information indicating a conflict of nutritional consumption with the first item.

2. The electronic device of claim 1, wherein the processor is further configured to:
determine whether the first item includes, based on the information of the first item, a nutritional benefit based on the information of the first item;
identify, based on the information of the first item, the second item based on a determination that the first item includes the nutritional benefit; and
display, on the display, the information of the second item including information indicating the nutritional benefit for consuming with the first item.

3. The electronic device of claim 1, wherein the processor is further configured to:
identify a body condition including at least one of an illness, a disease, a pregnancy, or a diabetes;
determine whether the first item includes, based on the information of the first item, a nutritional restriction or benefit associated with the body condition based on the information of the first item; and
display, on the display, information indicating whether the first item includes the nutritional restriction or benefit of consumption for the body condition.

4. The electronic device of claim 3, wherein the processor is further configured to:
identify a medicine being used for the body condition;
determine whether the first item includes, based on the information of the first item, the nutritional restriction or benefit associated with the medicine based on the information of the first item; and
display, on the display, information indicating whether the first item includes the nutritional restriction or benefit of consumption for the medicine.

5. The electronic device of claim 1, wherein the processor is further configured to:
identify an age condition;
determine whether the first item includes, based on the information of the first item, a nutritional restrictions or benefits associated with the age condition based on the information of the first item; and
display, on the display, information indicating whether the first item includes the nutritional restrictions or benefits for the age condition.

6. The electronic device of claim 1, wherein the processor is further configured to:
identify an eating habit;
determine whether the first item includes, based on the information of the first item, a nutritional restriction for the eating habit based on the information of the first item; and
display, on the display, information indicating whether the first item includes the nutritional restriction for the eating habit.

7. The electronic device of claim 1, wherein the processor is further configured to:
determine whether the first item includes, based on the information of the first item, an intake cycle for consuming of the first item; and
display, on the display, information indicating the intake cycle of the first item for healthy consuming of the first item.

8. The electronic device of claim 1, wherein:
the memory is configured to:
store at least one of a pre-determined body condition, a pre-determined age condition, or a pre-determined eating habit; and
store at least one of a body condition, an age condition, or an eating habit being entered through an input circuit including at least one of a touchscreen or a key pad of the electronic device; and
the input readable circuit includes:
a camera that is capable of reading at least one of a QR code or a bar code associated with the information of the first and second items; or
at least one of a quick response (QR) code reader or a bar code reader associated with the information of the first and second items.

9. A method of an electronic device for controlling nutritional consumption, the method comprising:
obtaining information of a first item;
determining the information of the first item;
saving the information of the first item in a memory;
determining whether the first item includes a nutritional restriction based on the information of the first item;
identifying, based on the information of the first item, information of a second item based on a determination that the first item includes the nutritional restriction, wherein the information of the second item is stored in the memory; and
displaying the information of the second item including information indicating a conflict of nutritional consumption with the first item.

10. The method of claim 9, further comprising:
determining whether the first item includes, based on the information of the first item, a nutritional benefit based on the information of the first item;
identifying, based on the information of the first item, the second item based on a determination that the first item includes the nutritional benefit; and
displaying, on the display, the information of the second item including information indicating the nutritional benefit for consuming with the first item.

11. The method of claim 9, further comprising:
identifying a body condition including at least one of an illness, a disease, a pregnancy, or a diabetes;
determining whether the first item includes, based on the information of the first item, a nutritional restriction or benefit associated with the body condition based on the information of the first item; and
displaying, on the display, information indicating whether the first item includes the nutritional restriction or benefit of consumption for the body condition.

12. The method of claim 11, further comprising:
identifying a medicine being used for the body condition;
determining whether the first item includes, based on the information of the first item, the nutritional restriction or benefit associated with the medicine based on the information of the first item; and
displaying, on the display, information indicating whether the first item includes the nutritional restriction or benefit of consumption for the medicine.

13. The method of claim 9, further comprising:
identifying an age condition;
determining whether the first item includes, based on the information of the first item, a nutritional restrictions or benefits associated with the age condition based on the information of the first item; and
displaying, on the display, information indicating whether the first item includes the nutritional restrictions or the benefits for the age condition.

14. The method of claim 9, further comprising:
identifying an eating habit;
determining whether the first item includes, based on the information of the first item, a nutritional restriction for the eating habit based on the information of the first item; and
displaying, on the display, information indicating whether the first item includes the nutritional restriction for the eating habit.

15. The method of claim 9, further comprising:
determining whether the first item includes, based on the information of the first item, an intake cycle for consuming of the first item; and
displaying, on the display, information indicating the intake cycle of the first item for healthy consuming of the first item.

16. The method of claim 9, further comprising:
storing at least one of a pre-determined body condition, a pre-determined age condition, or a pre-determined eating habit;
storing at least one of a body condition, an age condition, or an eating habit being entered through an input circuit including at least one of a touchscreen or a key pad of the electronic device;
obtaining, through a camera, the information of the first and second items based on at least one of a QR code or a bar code; and
obtaining, through a quick response (QR) code reader or a bar code reader, the information of the first and second items based on at least one of a QR code or a bar code associated with the information of the first and second items.

17. A non-transitory computer-readable medium comprising program code, that when executed by at least one processor, causes an electronic device to:
obtain information of a first item;
determine, through an input readable circuit, the information of the first item;
save the information of the first item in a memory;
determine whether the first item includes a nutritional restriction based on the information of the first item;
identify, based on the information of the first item, information of a second item based on a determination that the first item includes the nutritional restriction, wherein the information of the second item is stored in the memory; and
display, on a display, the information of the second item including information indicating a conflict of nutritional consumption with the first item.

18. The non-transitory computer-readable medium of claim 17, further comprising program code, that when executed by at least one processor, causes an electronic device to:
determine whether the first item includes, based on the information of the first item, a nutritional benefit based on the information of the first item;
identify, based on the information of the first item, the second item based on a determination that the first item includes the nutritional benefit; and
display, on the display, the information of the second item including information indicating the nutritional benefit for consuming with the first item.

19. The non-transitory computer-readable medium of claim 17, further comprising program code, that when executed by at least one processor, causes an electronic device to:
identify a body condition including at least one of an illness, a disease, a pregnancy, or a diabetes;
determine whether the first item includes, based on the information of the first item, a nutritional restriction or benefit associated with the body condition based on the information of the first item; and
display, on the display, information indicating whether the first item includes the nutritional restriction or benefit of consumption for the body condition.

20. The non-transitory computer-readable medium of claim 19, further comprising program code, that when executed by at least one processor, causes an electronic device to:
identify a medicine being used for the body condition;
determine whether the first item includes, based on the information of the first item, the nutritional restriction or benefit associated with the medicine based on the information of the first item; and
display, on the display, information indicating whether the first item includes the nutritional restriction or benefit of consumption for the medicine.
